(19) United States
(12) Patent Application Publication
(10) Pub. No.: US 2015/0037388 A1
(43) Pub. Date: Feb. 5, 2015

(54) EDIBLE SLOW DISSOLVING FILM FOR TREATING ORAL ULCERATIONS

(71) Applicant: BETA PHARMA S.A., Panama City, PA (US)

(72) Inventor: Sonia Longo, Sesto S. Giovanni (IT)

(21) Appl. No.: 14/374,814

(22) PCT Filed: Jan. 28, 2013
(86) PCT No.: PCT/EP2013/051573

§ 371 Date: Jul. 25, 2014

(30) Foreign Application Priority Data
Jan. 27, 2012 (EP) PCT/EP2012/051307

(51) Int. Cl. A61K 35/64; A61K 47/32; A61K 47/38; A61K 47/36; A61K 31/12; A61K 9/00

(52) U.S. Cl. CPC A61K 35/644; A61K 31/12; A61K 9/0056; A61K 47/38; A61K 47/36; A61K 47/32
USPC 424/439; 514/779; 424/539; 514/679

(57) ABSTRACT

The invention relates, in one aspect, to a mucoadhesive edible slow dissolving film comprising a salt of alginic acid, an edible cellulose derivative, an edible protein, and edible polymer delaying the dissolution in a physiological fluid of the edible film selected from polyvinylpyrrolidone, a carboxyvinyl polymer and mixtures thereof, in an amount effective to delay the dissolution time of the film in a biological fluid. The edible film is suitable for cicatrizing oral ulcerations or lesions or for forming a layer on oral mucosae or gingiva affected by ulcerations or lesions for protecting against mechanical abrasions, infections or caustic agents.

### FIELD OF THE INVENTION

The present invention relates to a mucoadhesive edible slow dissolving film for protecting, treating oral ulcerations and lesions.

The present invention origins in the medical filed and in particular in the field of medical products or remedies intended for the use in the cure, mitigation, treatment, or prevention of oral ulcerations.

Specifically, the present invention concerns an edible film having a low dissolution rate in biological liquids, intended to speed up the time for cicatrizing or re-epithelizing lesions of the oral mucosa.

### PRIOR ART

Most of the mucosa lining the oral cavity, such as floor of the mouth, cheeks, ventral tongue, is thin and delicate, rendering it susceptible to trauma. By contrast, the mucosa of the hard palate and gingiva is keratinised and more resistant to injuries.

Any injuries to the mucosa of the oral cavity may result in a defect or damage of the surface in which the covering epithelium is damaged, leaving an inflamed area of exposed connective tissue. These damages are commonly called ulcers or erosions.

Typically, the oral ulcer is caused by the complete loss of epithelium accompanied by variable loss of the underlying connective tissue, which results in a crateriform appearance. It may be augmented by oedema and/or proliferation of the surrounding tissue.

Usually oral ulcerations are transient and self-resolving that is tend to resolve within a few days without the need for medical or dental intervention.

However, most of them may require at least a self-made medication.

Some of the ulcers do not heal within 2 weeks and may be considered persistent or chronic. These require self-medication or the intervention of the medical or dental practitioner.

Oral ulcerations have been classified for obtaining a proper diagnosis and directing the patient or the clinician toward appropriate therapy.

A first classification system of oral ulcerations is based on distinguishing whether the ulceration is simple, complex, or destroying.

An oral ulceration is defined simple when a single ulcer occurs without the involvement of the remaining mucosa.

Complex ulcerations include single or multiple ulcers with changes to the surrounding mucosa, skin, and/or systemic manifestations. The lesion may be white, red, or vesiculobullous.

Destroying ulcerations are diffuse lesions with tissue destruction and severe systemic involvement.

Oral ulceration may origin from different causes. The main causes are of traumatic, infective, idiopathic or neoplastic origins. In some cases oral ulcerations are associated with dermatological or systemic diseases. In fact, the biologically dynamic nature of the oral mucosa makes it vulnerable to the effects of systemic disease.

Most of the oral ulcerations have a traumatic origin. Amongst traumatic lesions, mechanical trauma caused by accidental biting, a sharp edge of a tooth, or by the use of ill-fitting intraoral devices such as dental plates are very common. These ulcers usually heal at a moderate speed if the source of the injury is removed, for example, if poorly fitting dentures are removed or replaced.

Some ulcers also occur after dental work, when incidental abrasions to the soft tissues of the mouth occur. A dentist usually apply a protective layer of petroleum jelly before carrying out dental work in order to minimize the number of incidental injuries to the soft mucosa tissues.

Usually, a traumatic ulcer shows the histological features of chronic unspecific inflammation.

Chemicals used in the dental practice or oral hygiene may be a further cause of oral ulcerations. In particular, oral ulcerations may occur due to the accidental application of caustic or disinfectant agents during dental practice or by the chronic use of preparations commonly used in self-treatment of oral complaints. In particular, the reiterated use of antiseptic mouthwashes or of oral disinfectants may determine the formation of oral ulcerations.

In these cases the reactions vary in severity from edema through localized necrosis of the epithelium.

Oral ulceration on any part of the oral mucosa may also occur with acute thermal trauma, for example when an individual takes very hot food or beverages.

In these cases ulcerations are usually present in the palate.

Bacterial, viral or fungal infections in the oral cavity may be an additional cause of oral ulceration. In particular, cold sores also mentioned as fever blisters, oral herpes, which origins around the lip are caused by viruses.

Ulcer may be also caused by neoplastic diseases. Ulcers occurring at the site of the original neoplasm may be difficult to differentiate from recurrent tumours.

Idiopathic causes may also causes the formation of ulcers with frequent recurrences over prolonged period of time.

Usually, this group of idiopathic ulcers is referred as recurrent aphthous stomatitis also known as RAS. Recurrent aphthous stomatitis affects 5% to 25% of the general population.

Three different types of ulcers are recognized, based on their clinical features: minor aphthous ulcers, major aphthous ulcers and herpetiform ulcers.

The treatments of these types, substantially, depend on the severity of the lesions. Symptomatic treatment is the primary approach to dealing with oral ulcers.

In mild cases, with one to three lesions, use of topical antihistamines, antacids, is appropriate.

Rinsing the mouth out with brine (warm salted water) or rubbing salt or garlic on the sore area may help to cure an ulcer.

Paracetamol or ibuprofen or of anesthetic agents such as benzocaine in the form of local anesthetic lozenges, paints or mouth rinses are also used to obtain pain relief. Avoiding spicy or hot foods is also recommended to reduce pain.

In more severe cases, the use of topical corticosteroid preparations, such as betamethasone, fluocinolone, or clobetasol places directly on the lesion is quite common to shorten healing time and the size of ulceration.

Ulcers persisting longer than three weeks may require the attention of a medical practitioner. In certain cases, aphthous ulcers are treated by Silver nitrate, Amlexanox paste.

However, the remedies and medicaments available up to date for treating and alleviating symptoms and pain connected with oral ulcerations have not been proved satisfactory.

Accordingly at present there is the need for new remedies suitable for alleviating the symptomatology accompanying ulcerations in the oral mucosa and or reducing recovery time.

One of the object of the present invention resides in the provision of a local remedy for alleviating the symptoms and pain accompanying oral ulcers.

An additional object of the present invention resides in the provision of a local remedy suitable for protecting ulcerations of the oral mucosa and accelerate the recovery time of the mucosa.

Yet an additional object of the present invention is the provision of a medical remedy in the form of a film or patch which can be easily applied on the oral mucosa affected by ulcerations as a self-medication.

### SUMMARY OF THE INVENTION

The inventor has found that the incorporation of a specific edible polymer selected from polyvinylpyrrolidone (PVP), a carboxyvinyl polymer and a mixture thereof in an alginate-based edible film, changes the rheological properties of the alginate-based film, prolongs its dissolution time in water or in a physiological liquid and enables the film to act as a physiological barrier.

The inventor has also found that the (combined) use of both polyvinylpyrrolidone (PVP) and a carboxyvinyl polymer results in a synergistic delay of the dissolution in a physiological fluid of the edible mucoadhesive film of the invention and in an unexpected long-lasting protection of the mucosae on which the film is applied.

The edible alginate-based film incorporating PVP, a carboxyvinyl polymer and preferably a mixture thereof, is applicable onto oral ulcerations where it acts as a long lasting barrier which protects the mucosa against external stresses of mechanical, physical or chemical origins, thus accelerating the physiological process of cicatrization of the mucosa.

In a first aspect the present invention thus provides a mucoadhesive edible slow dissolving film for protecting and/or treating oral ulcerations comprising a salt of alginic acid, a cellulose derivative, an edible protein and an edible polymer delaying the dissolution in a physiological or biological fluid of the edible film selected from polyvinylpyrrolidone, a carboxyvinyl polymer and mixtures thereof, in an amount effective to slow or delay the dissolution time of the film in water or a biological fluid.

Typically, the edible polymer delaying the dissolution in water of the edible film is present in the formulation of the edible film in an amount effective to reduce the solubility of the alginate-based film in a biological liquid, saliva in particular.

In particular, the presence of polyvinylpyrrolidone, a carboxyvinyl polymer or a mixture thereof in the edible solid film according to this first aspect of the invention increases the dissolution time of the film in a biological liquid and increase the adhesive effect of the film to the oral mucosa.

Thus, the presence of the selected edible polymer delaying the dissolution in water of the edible film avoids that the edible solid film rapidly dissolves in the saliva of the oral cavity, and increases the time of adhesion of the edible solid film to the mucosal ulceration and the protective effect against potential traumatic damages. Typically, the dissolution time of the film remains substantially the same when calculated in water or in a biological liquid such as saliva.

In certain embodiments the mucoadhesive edible slow dissolving film of the invention has a dissolution time in water at 37° C. of 1 minute to 6 hours, typically from 20 minutes to 3 hours. A remarkable increased dissolution time of 40 minutes to at least 2 hours is obtained using a synergistic combination of PVP and carboxyvinyl polymer.

When the mucoadhesive edible slow dissolving film of the invention is applied to a mucosal area affected by ulceration, it firmly adheres to the oral mucosa making a barrier which protects the ulceration from traumatic events and/or from bacterial, viral or fungal infections.

According to certain embodiments of the invention the mucoadhesive edible slow dissolving film of the invention is or forms a physical barrier to mechanical injuries or abrasions of mucosae or gingiva caused, for example by the mastication of food or by teeth during mastication of ingested substances.

In certain preferred embodiments of the edible film of the invention both the polyvinylpyrrolidone and the carboxyvinyl polymer are present to provide a synergistic delay of the dissolution rate of the edible film.

For the purposes of the present invention the terms “slow dissolving film”, “film with delayed dissolution” and “delayed dissolution film” have the same meaning. In a second aspect the invention relates to an mucoadhesive edible slow dissolving film comprising a salt of alginic acid, a cellulose derivative, an edible protein, an edible polymer delaying the dissolution in water of the edible film selected from polyvinylpyrrolidone, a carboxyvinyl polymer and mixtures thereof and at least one selected active ingredient which provides a cicatrizing and/or re-epithelialization synergistic effect of the oral ulcers/ulcerations/lesions of the oral cavity.

In accordance with certain embodiments of this second aspect, the inventors has found that when two selected active ingredients of natural origins are incorporated in the formulation of the edible film of the invention, a synergistic re-epithelialization and cicatrizing effect is achieved on the oral mucosa affected by ulcers, lesions.

In certain embodiments of the present invention the selected active ingredients of natural origins are propolis and a curcuminoid. The combination of propolis with a curcuminoid provides a synergistic cicatrizing effect on oral ulcerations which speed up the re-epithelialization of the oral mucosa affected by ulcerations reducing the recovery time.

The mucoadhesive edible slow dissolving film according to this aspect of the invention is specifically suitable for use in a method of treatment of lesions of the oral mucosae, such as aphthous ulcers and/or herpetic forms.

Thus, in accordance with a third aspect of the invention it is provided the use of a mucoadhesive edible slow dissolving film comprising, a salt/derivative of alginic acid, a cellulose derivative, an edible protein, an edible polymer delaying the dissolution in water of the edible film selected from polyvinylpyrrolidone, a carboxyvinyl polymer and mixtures thereof, and optionally water, for protecting oral ulcers/ulcerations against the action of mechanical, infective or caustic agents.

In certain embodiments the oral ulceration is a lesion of the oral mucosa such as an aphthous ulcer or an herpetic ulcer, RAS.

In certain embodiments, the present invention provides a mucoadhesive edible slow dissolving film comprising a salt or derivative of alginic acid, a cellulose derivative, an edible protein, polyvinylpyrrolidone, a carboxyvinyl polymer, propolis, and a curcuminoid, optionally water for the use for cicatrizing or re-epithelizing oral ulcerations.

In accordance with another aspect of the invention a method for protecting oral mucosae, gingiva, lesions, oral ulcerations from mechanical, chemical, infective agents or injuries is hereby provided, said method comprising the application of a mucoadhesive edible slow dissolving film according to anyone of the embodiments herein described, to an area of oral mucosa or to an oral ulceration of a subject.

In accordance with this aspect, the mucoadhesive edible slow dissolving film of the invention when applied on an area of oral mucosa, gingiva, oral ulcerations or lesions makes a physical barrier which protect the mucosa or gingiva from aggression of the agents, such as bacterial infiltrations in the gingiva, and/or prevents from accidental abrasion caused by teeth during mastication of an edible substance. The formation of a physical barrier for a prolonged time is particularly suitable for preventing the formation of ulcerations or aphthae, in particular in young people.

### DESCRIPTION OF THE FIGURES

The present invention will now be described with reference to the following examples which are provided for the purpose of illustration and are not intended to be construed as being limiting on the present invention, and further with reference to the figures as described briefly below.

FIG. 1 shows the ability of an edible gel of Example 1 to adhere to a mucosa. The adhesion abilities on mucosa of gels with or without the edible polymer delaying the dissolution in water of the film, are compared.

FIG. 2 shows the inhibition of an edible gel containing propolis and a curcuminoid as active ingredients on the growth of a bacterial culture.

### DETAILED DESCRIPTION OF THE INVENTION

In accordance with the first aspect of the present invention a mucoadhesive edible film is provided including a cellulose derivative, a salt of alginic acid, an edible protein and optionally water, as basic components and edible polymer delaying the dissolution in water of the edible film selected from polyvinylpyrrolidone, a carboxyvinyl polymer and mixtures thereof as component suitable for delaying the dissolution rate of the edible solid film in a biological liquid, saliva typically.

In accordance with a preferred embodiment of the invention the mucoadhesive edible film contains a synergistic mixture of polyvinylpyrrolidone and carboxyvinyl polymer. The protective synergistic effect on oral mucosae, gingival, oral ulcerations or lesions and the prolonged time of dissolution in water at room temperature obtained by using a mucoadhesive film containing a combination of polyvinylpyrrolidone and carboxyvinyl polymer is demonstrated in the experimental part contained in the following Examples 7-9. The use of a mixture of polyvinylpyrrolidone and carboxyvinyl polymer in the formulation of the mucoadhesive edible film has the advantage of prolonging the dissolution time of the film in a physiological fluid and of increasing the comfort and compliance.

For purposes of the present application, the term “edible” is intended to mean food grade materials which are approved by regulatory authorities for use in pharmaceutical or food applications.

Typically, the edible film of the invention is an alginate-based film. Thus, a salt of alginic acid is a basic component of the edible film of the invention. Alginic acid is a well-known anionic polysaccharide which is available from the cell walls of brown algae, where it, through binding water, forms a viscous gum.

In certain embodiments the polymeric alginate is provided with a MW of 1×10³ to 1×10⁸ daltons and a viscosity of 300 to 900 cps, measured in an aqueous solution of 1% by weight.

Suitable salts of alginic acid are physiologically acceptable salts of alginic acid with particular reference to salts with alkali metal or alkaline earth metals.

In certain embodiments of the invention calcium, magnesium or/and sodium salts of alginic acid are used. These salts may be found, for example, in the cell walls of brown algae.

In certain embodiments the salt of alginic acid is sodium alginate.

Typically, the salt of alginic acid is present in the mucoadhesive edible slow dissolving film in an amount of 20 to 65% by weight, preferably of 25 to 50% by weight of the film.

The edible polymer delaying the dissolution in a physiological liquid of the edible film is an ingredient of the film of the invention.

Typically, the term physiological or biological liquid/fluid means water or a liquid naturally occurring in the human body, for example saliva.

A suitable edible polymer delaying the dissolution in a physiological fluid (hereinafter also referred to as the edible polymer) is selected from polyvinylpyrrolidone, a carboxyvinyl polymer and mixtures thereof. In certain embodiments the edible polymer is present in an amount of 0.1 to 20%, typically from 1 to 10% by weight.

In certain embodiments where the edible polymer content is in the range of 1 to 10% by weight and the film has a thickness in the range of 100 to 200 μm, the dissolution rate of the film is of 20 minutes to 2 hours. This dissolution time exceed of a great extent the dissolution time of 20 seconds to 3 minutes of an alginate based film with the same composition wherein the edible polymer is absent.

In certain embodiments of the invention polyvinylpyrrolidone is used as edible polymer to slow the dissolution time of the alginate-based film in a biological fluid. Typically, polyvinylpyrrolidone is present in the edible film in an amount effective to slow or delay the dissolution time of the film in a physiological fluid such as saliva. The terms slow dissolving film means that the film of the invention has a dissolution or solubilization time in water at 37° C. which is greater than the dissolution/solubilization time of a conventional film containing sodium alginate of 25 to 60%, microcrystalline cellulose of 0.1 to 20% by weight and vegetable proteins of 0.1 to 25% by weight such as those disclosed in EP 2 151 252A2, whose content is hereby fully incorporated by reference.

Typically, an average dissolution time, of 20 minutes to 2 hours, has been measured by application of a film of 2.5 cm² and having a thickness of 0.02 cm, to oral mucosa of an individual. In these conditions, a dissolution time of 31 min has been measured using the film of Example 8.

In certain embodiments polyvinylpyrrolidone is present in the edible film in an amount of 0.1 to 25% by weight, preferably of 0.5 to 15% by weight, more preferably of 1 to 5% by weight.

In certain embodiments the polyvinylpyrrolidone has a molecular weight of 1×10³ to 1×10⁹ daltons, preferably from 1×10⁴ to 1×10⁷ daltons.

In certain embodiments a carboxyvinyl polymer is used as edible polymer to slow the dissolution time of the alginate-based film in a biological fluid.

A suitable carboxyvinyl polymer is a water soluble high molecular weight polymer of acrylic acid cross-linked with various crosslinking agents, in particular polyalkenyl (poly)ethers, divinyl glycol or divinyl benzene. For example suitable polyalkenyl (poly)ethers are polyallylsucrose, polyallylpentaerythritol

In certain embodiments, the carboxyvinyl polymers have a molecular weight of 20,000 to three millions. In certain embodiments the carboxyvinyl polymers have a viscosity which may range from 1000 to 7000 centipoises (cP).

Typically, a very high viscosity can be obtained by adding a small amount of carboxyvinyl polymers such as of 0.1 to 5% by weight of the film weight.

Suitable carboxyvinyl polymers include those having the generic name of carbopol or carbomer. Typical examples are Acrylates/C10-C30, Alkyl Acrylates Cross polymer.

A suitable carbopol is carboxypolymethylene.

For example the Carbopol 971P NF polymer manufactured by Lubrizol, Cleveland, Ohio, in the USA, is a suitable edible polymer.

The three dimensional nature of these polymers confers some unique characteristics, such as biological inertness, not found in similar linear polymers.

In particular, it has been found that the addition of a carboxyvinyl polymers is suitable for increasing the bioadhesion of the edible film to the oral mucosa. In addition a carboxyvinyl polymer, when incorporated in the edible film of the invention, is suitable for the controlled release of the active ingredients.

In certain embodiments the carboxyvinyl polymers is present in an amount of 0.01 to 10% by weight, preferably of 0.5 to 5% by weight.

In certain embodiments of the edible film of the invention both the PVP and the carboxyvinyl polymer are present. In these embodiments the two edible polymers exert a synergistic delay of dissolution in water of the edible film. In addition the presence of both the PVP and the carboxyvinyl polymer exerts a synergistic adhesive effect of the edible film on oral mucosa.

An edible cellulose derivative is also a basic component of the edible film of the invention.

For the purposes of the present application the term edible cellulose derivatives includes chemically modified forms of cellulose commonly used in food processing and pharmaceutical industry.

Suitable cellulose derivatives may be selected among hydroxypropylmethyl cellulose, methyl cellulose, hydroxypropyl cellulose, hydroxylethyl cellulose, carboxymethyl cellulose, ethylhydroxyethyl cellulose, cellulose acetate butyrate, cellulose acetate phthalate, hydroxypropyl-methylcellulose, ethyl-methylcellulose and mixtures thereof.

In certain embodiments the edible cellulose derivative is microcrystalline cellulose, a partially depolymerized cellulose usually in the form of a crystalline powder.

In certain embodiments the edible cellulose derivative, in particular microcrystalline cellulose, is present in the slow dissolving film in an amount of 0.1 to 25% by weight, preferably of 1 to 10% by weight.

In certain embodiments, water is also contained in the edible film of the invention. In certain embodiments the edible film contains water, in particular in an amount such that its activity (Aw), that is the ratio of the vapor pressure of the water contained in the mixture, in particular in the solid form, to the vapor pressure of pure water at the same temperature is of 00.5 to 0.6, preferably of 0.1 to 0.3, where the film conveniently has a thickness of 50 to 200 μm.

In certain embodiments, the mucoadhesive edible slow dissolving film of the invention contains edible proteins.

Suitable edible proteins are those which are commonly used as emulsifiers or aggregating agent in the food or dietary/pharmaceutical industry. Suitable proteins may be of vegetal or animal origins, those of vegetable origins being preferred. Suitable animal protein are albumins, or proteins derived from fish.

In certain embodiments the protein is of vegetal origin and typically origins from cereals or legumes for example from peas.

The presence of edible proteins of animal or vegetal origins contributes to the mechanical properties of the film and in certain embodiments contributes to maintain an Aw value of 00.5 to 0.6, preferably of 0.1 to 0.3.

In certain embodiments the edible proteins are contained in the edible film in an amount of 0.1 to 25%, preferably of 1 to 10% by weight of the total film weight, typically after solvent evaporation.

In certain embodiments, the composition of the invention further includes one or more additional components, such as additives, fillers, stabilizers, emulsifying, texturizing, filmogenic, plasticizing, wetting agents, and thickeners.

In certain embodiments the edible film of the invention contains one or more emulsifying agents commonly used in the food or dietary field.

Suitable emulsifying agents include polyoxyethylene derivatives for example polysorbates, natural gums such as carob seed flour or acacia gum, and/or gelatins.

Typically, emulsifying agents, when added to the film composition of the invention, may be present in an amount of from 0.1 to 15% by weight of the total weight.

In certain embodiment one or more wetting agents are present in the edible film of the invention. Suitable wetting agents are polyols, such as glycerin, propylene glycol, sugar alcohols which are commonly used in the food industry such as maltitol, sorbitol, xylitol and/or isomalt or ester of carboxylic acid such as acetic acid with glycerol.

In certain embodiments texturizing agent are incorporated in the edible film of the invention. Suitable texturizing agents includes xanthan gum and/or Arabic gum. For example texturizing agents may be present in amount of 0.′1 to 3%, preferably of 0.1 to 1% by weight.

In certain embodiments of the invention, wetting agents may be present in an amount ranging from 0.1 to 50% by weight, preferably from 0.5 to 5% by weight.

In certain embodiment the edible film of the invention contains one or more emulsifying agents. Suitable emulsifying agent are starches or starch derivatives commonly used in the food or dietary industry typically from maize, potatoes.

In certain embodiments the edible film of the invention further contains a polysaccharide, in particular hyaluronic acid. This is a polysaccharide with alternating 1-3 glucuronidic and 1-4 glucosaminidic bonds. The Applicants have found that the incorporation of hyaluronic acid or its derivatives into the edible film increases the adhesion time of the film to the mucosae.

Moreover, the hyaluronic acid keeps the edible film in close contact with the mucosa and extends the time of release of any active principles contained in the film.

An additional benefit obtained by the presence of hyaluronic acid or its derivatives in the edible film of the invention derives from the healing effect they display on a bleeding mucosa.

In certain embodiments, hyaluronic acid is present in the film in the form of a salt, for instance as the sodium or potassium salt, or as a derivative or complex with other components, for example with glucosamine.

In certain embodiments hyaluronic acid or a salt thereof is present in the film in an amount of 0.1 to 10% by weight, preferably of 0.5 to 5% by weight.

In certain embodiments the mucoadhesive edible slow dissolving film has a thickness of 50 to 800 μm, preferably of 70 to 500, more preferably of 90 to 200 μm.

In certain embodiments the edible film of the invention has rectangular or square forms and dimensions depending from the extension of the lesion to be treated or covered.

Typically the edible film of the invention is applied directly on the oral ulcers in order to make a protective layer or a polymeric barrier which prevents additional damages of the damaged oral mucosa.

In certain embodiments the edible film of the invention is in the form of a patch for the application on diseased oral mucosae. The patch may be applied directly on oral mucosae affected by ulcerations for protecting against mechanical abrasions, infections or caustic agents.

In accordance with certain embodiments of the present invention, the mucoadhesive edible slow dissolving film further contains one or more active ingredients.

Within the present invention the term “active ingredient” refers to any substances having pharmaceutical or pharmacological effect or to any biologically active substance. Biologically active substance refers to a substance that, once applied or administered topically, displays some kind of biologic action. Typical examples of biologically active substances are vitamins, natural extracts, physiologically acceptable mineral salts. The active ingredients may have disinfectant or antiviral activity, and/or cicatrizing activity on oral mucosal lesions. Suitable active ingredients are for example those having antibacterial activity commonly used in the preparation of formulation for oral hygiene, such as chlorhexidine, cationic surfactants, alcohol, etc.

In certain embodiments of the invention the edible film may contain a combination of active ingredients. Within the invention any combination of topically suitable active principles can be used.

In accordance with the second aspect of the present invention the mucoadhesive edible slow dissolving film of the invention contains an association of selected active ingredients of natural origins: propolis and a curcuminoid.

Curcuminoids are substances naturally occurring in Turmeric (Curcuma longa L.), the popular Indian spice which is a major ingredient of curry powders.

In particular, the turmeric extract contains three major curcuminoids: curcumin, demethoxycurcumin, bisdemethoxycurcumin. All the three curcuminoids can be used for the purposes of the present invention, curcumin being the preferred.

In certain embodiments suitable curcuminoids are those formulated and marketed under the trade-name of MERIVA® by Indena S.p.A.—Milan, Italy.

In certain embodiment one or more curcuminoids are incorporated in the edible film of the invention in an amount of 0.01 to 10% by weight, preferably of 0.5 to 5% by weight.

The other suitable active ingredient is propolis, a resinous mixture that honey bees collect from tree buds, sap flows, or other botanical sources. Propolis is sticky at and above room temperature whereas at lower temperatures, it becomes hard and very brittle. For its intrinsic properties, propolis can be easily incorporated in the formulation of the edible film of the invention.

Propolis in the edible film of the invention, exerts antimycotic, antifungine, antibacterial actions. In addition propolis also exerts anesthetic, vasoprotective, cicatrizing and re-epithelizing activity on the oral mucosae affected by ulcerations. In certain embodiment propolis is incorporated in the edible film of the invention in an amount of 0.01 to 10% by weight, preferably of 0.5 to 5% by weight.

The inventors have found that when the edible film of the invention contains both propolis and a curcuminoid, a synergistic cicatrizing and re-epithelizing effect is exerted on oral ulceration.

With the term of “oral ulceration/ulcer” is meant any lesions of the oral mucosa including aphthae, herpetic forms, or their symptoms, included RAS.

In particular, the incorporation of the two selected ingredients in the edible film of the invention for example in an amount of 0.5 to 3% by weight, reduces the cicatrizing time of around 30%, when compared to an edible film with the same composition in which the two active ingredients are absent.

A surprising cicatrizing effect may be obtained when the film containing the association of the two active ingredients is maintained in contact with the ulceration of the oral mucosa for a suitable time which typically is greater than 10 minutes.

The application of the edible film directly on the mucosal ulceration together with the slow dissolution of the film permits to maintain the combination of the two active ingredients on the oral ulcerations and to exerts the recovery of the oral mucosa. In these conditions, an unexpectedly accelerated cicatrizing effect is achieved by the edible film of the invention.

In accordance with other aspects, the present invention relates to the above edible film for use in the treatment or prophylaxis of oral ulcerations, in particular aphthous ulcers, herpetiform ulcers, recurrent aphthous stomatitis (RAS) and other ulcerations in the palate, tongue and annexes tissues.

The edible film of the invention may be applied to the oral mucosa in need of treatment in a “acceptable amount”, this being an amount sufficient to at least stop the progression of the ulceration in the oral mucosa.

Alternatively, the edible film may be administered to a subject in need of treatment to a “therapeutically effective amount” this being an amount sufficient to show benefit to the subject. In particular the benefit may be the amelioration of at least a symptom associated with the condition of the oral mucosa to be treated or the prevention or partial inhibition of the onset of at least one symptom associated with the condition. The severity and or time of onset of at least one symptom may be reduced with the application of the composition.

Typically, the actual amount and the rate and time course of administration of the composition will depend on the nature and severity of the condition to be treated. Prescription of application such as decisions on frequency of application is within the discretion of the patient and depends on the severity of the symptoms.

In certain embodiments the composition is applied to the oral mucosa in need of treatment one to four times a day.

The natural origins of the active ingredients makes the edible film also indicated for pediatric applications.

In accordance with yet another aspect a method for preventing the formation or for the treatment of oral ulcerations, lesions such as aphthae is provided, said method comprising the application of a mucoadhesive edible polymeric film according to any of the above embodiments to an area of oral mucosa of a subject.

The edible film of the invention can be produced according to procedure which are commonly used in the field of edible films such as those described in EP 2151252 which is incorporated here by reference.

For example the components of the film are charged in a suitable mixer to which water is added. The water conveniently is pre-heated at a suitable temperature such as in the range of 45 to 75° C.

The basic components comprising the alginate, the cellulose derivative and PVP are trickled in under stirring in order to give an homogeneous mixture and prevent the formation of lumps.

The mixture is then cooled at a temperature which typically is in the range of 25 to 35° C. The obtained mixture is spread by a suitable blade as a film having a thickness for example of 150 to 300 μm, onto a silicone coated surface of a support belt designed to pass through a ventilation tunnel oven, provided with heating stations with temperatures up to around 125° C. and with the last station at the lowest temperature of 60 to 90° C. At the oven outlet a film having a thickness between 50-200 μm separates from the support belt and is cooled up to the environmental temperature, then punched into a suitable shape and size and finally divided into unit portions.

In certain embodiments the heated tunnel oven is provided with one or more drying stations which are heated at different temperatures. For example the heating temperature in the first station is in the range of 60 to 80° C., the temperature in the second and third stations is in the range of 70 to 90° C. and in the final temperature in the range of 60 to 80° C.

The present application claims the priority of the International application No. PCT/EP2012/051307 of 27 Jan. 2012, whose content is hereby fully incorporated by reference.

The following examples are provided merely for illustrating the present invention and are not to be intended as limiting the scope of protection as results from the appended claims.

### Example 1

An edible film with cicatrizing activity has been formulated with the following composition:

| | |
|---|---|
| Sodium alginate | 23.64% |
| Mycrocristalline cellulose | 8.04% |
| Polyvinylpyrrolidone | 4.72% |
| Carbomer | 1.42% |
| Vegetal proteins from pea | 14.18% |
| Glycerin 99.5% | 23.64% |
| Sorbitol | 8.27% |
| Polysorbate 80 | 9.46% |
| Curcumin | 1.18% |
| Propolis | 0.71% |
| Aroma | 2.36% |
| Sodium hyaluronate | 2.36% |

### Example 2

An edible film for covering ulcers, acting as a protective barrier against mechanical abrasions has been manufactured with the following composition

| | |
|---|---|
| Sodium alginate | 40% |
| Mycrocristalline cellulose | 15% |
| Polyvinylpyrrolidone | 9% |
| Vegetal proteins | 1% |
| Glycerin 99.5% | 25% |
| Polysorbate 80 | 7% |
| Aroma | 3% |

### Example 3

An edible film applicable on aphthous or herpetic forms has been prepared with the following components

| | |
|---|---|
| Sodium alginate | 24.64% |
| Mycrocristalline cellulose | 7.04% |
| Polyvinylpyrrolidone | 4.72% |
| Carbomer | 0.42% |
| Vegetal proteins from pea | 15.18% |
| Glycerin 99.5% | 23.64% |
| Sorbitol | 8.27% |
| Polysorbate 80 | 9.46% |
| Curcumin | 1.08% |
| Propolis | 0.51% |
| Chlorexidin or acyclovir | 0.2% |
| Aroma | 2.25% |
| Sodium hyaluronate | 2.36% |

### Example 4

### Method of Manufacture of an Edible Film

The following components were used:

| | | |
|---|---|---|
| 1) Water (solvent) | 80 | Kg |
| 2) Sodium Alginate (Filmogen) | 7.0 | Kg |
| 3) Microcrystalline Cellulose (Thickener) | 0.4 | Kg |
| 4) Polyvinylpyrrolidone (Adhesive agent) | 16 | Kg |
| 5) Carbomer | 1.6 | Kg |
| 6) Vegetable Proteins (Emulsifiers/Texturizers) | 1 | Kg |
| 7) Polysorbate-80 (Emulsifier) | 1.5 | Kg |
| 8) Glycerin (Wetting agent) | 4.0 | Kg |
| 9) Sorbitol 70% (Plasticizer) | 2.0 | Kg |
| 10) Vanilla Flavour (Flavouring) | 1.0 | Kg |
| 11) Hyaluronic acid (Filmogen) MW 1,200,000 | 0.6 | Kg |

Method

The following steps were carried out:

In a first step polysorbate 80, glycerin, Sorbitol 70% and hyaluronic acid were dispersed under agitation in a mixer containing water preheated between 45° C. and 75° C. under continuous stirring until a clear lump-free gel is obtained.

Sodium alginate, microcrystalline cellulose, PVP, carbomer (Carbopol 971P NF) and vegetable proteins were trickled in and stirred for 35 min with slow agitation until dispersion is complete and the gel is homogeneous.

The homogeneous resulting mixture was cooled under stirring to 23-27° C., the vanilla flavor was added to the mixture; agitation is continued until homogeneous. The mixture obtained is fed onto a blade heated to 28-30° C. and then filmed to 200 μm thickness on a belt support of silicon-coated polyester. During film formation an aerated tunnel oven is used provided with four successive heating stations having the following increasing temperatures respectively: 65/70/75/80° C.

At the end of the oven tunnel a film of around 69 μm thickness (following water evaporation) spontaneously separates from the polyester support.

The film is then punched into patches having dimensions of 2×1.5 cm using a roller punch and having the following features

Weight of one unit (dimensions 2×1.5 cm): 38 mg
Thickness: 69 mAw. The water activity, i.e. the ratio of vapor pressure of the water present in the composition and the pressure of pure water at the same temperature: 0.1

The manufactures edible gel is applicable directly on aphtae and acts as a barrier against potential injuries and prevents the developments of infections.

### Example 5

Comparative adhesion tests on mucosa.

The adhesion times on gastric mucosa of 4 samples of edible films having the formulation of Example 1 and a thickness of 50 μm were compared with 4 sample of a reference film having the following formulation:

Reference film

| | |
|---|---|
| Sodium alginate | 26.64% |
| Mycrocristalline cellulose | 9.72% |
| Vegetal proteins from pea | 15.40% |
| Glycerin 99.5% | 25.24% |
| Sorbitol | 8.27% |
| Polysorbate 80 | 9.66% |
| Aroma | 2.26% |
| Sodium hyaluronate | 2.36% |

### Experimental Conditions

### Animals Characterisation

### Species: White Mice

### Strain: CD1

### N.: 5

Weight: 20-25 g at the starting of the test
Age: 8 weeks
Sex: female

### Caging

Each mouse was caged in cages of mm 160×270×370 (L×W×H).

Automatic control assured 12 hours light, 12 hours dark.

Room temperature and humidity were regulated by a conditioning plant.

### Cleaning and Disinfection

The cages and the housing room were cleaned before the animals were accommodated, then cleaning and disinfection were performed daily.

### Feeding

Animals were fed with standard pelletized diet, provided by Harlan Italy.

### Watering

Animals are watered with purified water, available ad libitum and distributed via an automatic watering system.

### Animal Identification

Animals selected for the study were identified via a numbered plastic mark applied to the right ear. Cages were identified with a tag.

### Animal Selection

Animals used in the study were randomly assigned to the different treatment groups at the beginning of the study.

### Materials and Methods

Gastric mucosa has been obtained from 8 week old mice. Immediately after sacrifice the stomach has been removed open longitudinally, the mucosa exposed and fixed on an hard by the use of needles. On the mucosa surface “the film of Example 1” and “Reference film” have been allowed to adhere, after that the samples have been dipped in a saline solution at 37° C. and gently steered for 3 and 10 minutes. After each time the samples were collected and frizzed at −20° C. to be cut at cryostat. Single section, 15 micron thick, have been collected and observed, unstained, under light microscopy. Images have been recorded using a digital camera.

### 2. Results

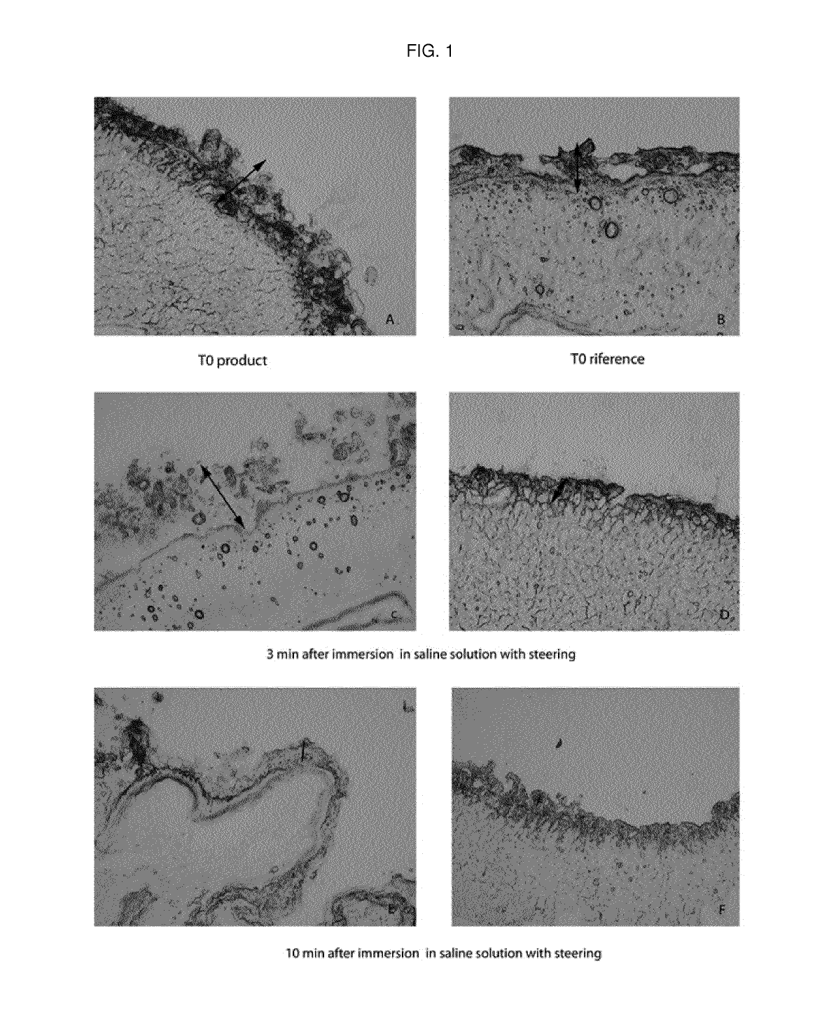

The images obtained under a light microscope (FIG. 1) clearly demonstrate an initial ability of both the films to adhere to a wet mucosa. Images A and B show respectively the film of Example 1 and the “reference film” immediately after their adhesion to the mucosa surface (no washing). Arrows indicates the products, to noteworthy the comparable thickness.

Images C and D show the mucosa surface with the films applied after 3 minutes of washing. Both films are still present on the mucosa surface, demonstrating their muco-adhesion ability. The “reference film” shows the lost of about 50% of thickness if compared to T0. At the contrary the “film of Example 1t” shows a thickens increase. Images E and F show the mucosa surface with the films applied after 10 minutes of washing. The “film of Example” is still visible on the mucosa surface, on the contrary the “reference film” is limited to a partially undetectable thin layer on the mucosa.

### Dissolution Times

Moreover the dissolution time has been evaluated for both the films. Both films, in their free form, have been dip in a saline solution at 37° C. and gently steered.

The “reference film” dissolved within 8/9 second (the evaluation has been performed in triplicate) versus about 90 minutes of the film of Example 1 including PVP (the evaluation has been performed in triplicate).

### Example 6

Objective of the study was to assess whether the edible gel having a thickness of 200 μm and the formula of Example 1 including the combination of propolis and a curcuminoid as active ingredients, exhibits a synergistic antimicrobial and cicatrizing effect.

For this aim a bacterial suspension (Micrococcus sp. 10 3 cfu/ml) was applied on a petri dish surface containing Muller Hinton medium. Subsequently about 1 square centimeter of the patch of Example 1 having a thickens of 100 μm was applied on top.

After 18 hours of incubation visual examination of the Petri dishes shows an inhibition of bacterial growth in contact with the gels. The bacteria grow was inhibited where in contact with components present in to the edible gel of Formula 1.

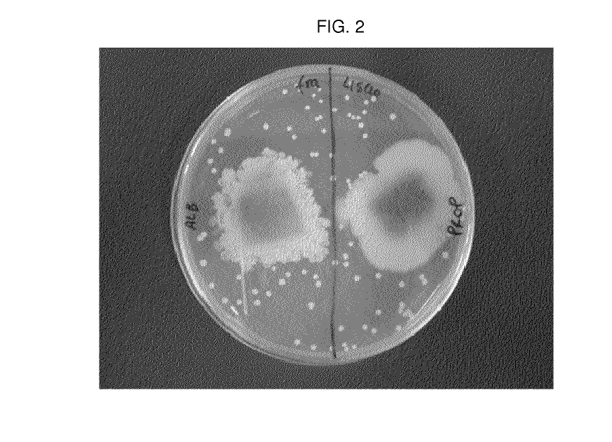

A representative photo of the analysis is reported in FIG. 2.

### Example 7

Mucoadhesive edible polymeric film according to invention, having a thickness of 50 μm and the following composition:

| | |
|---|---|
| Protanal GP 2650 (sodium alginate) | 30.33% |
| Avicel ph 105 (microcrystalline cellulose) | 6.30% |
| Pisane M9 (Vegetal proteins from pea) | 11.42% |
| Sorbitol 70% | 21.66% |
| Zemea | 5.51% |
| Tween 80 (Polysorbate 80) | 9.45% |
| Renovhyal (ialuronic acid) | 0.06% |
| Taurina | 0.59% |
| PVP K90 | 5.51% |
| Xilitol | 0.59% |
| Carbopol 971 P NF | 2.76% |
| Propoli | 0.59% |
| Symrise | 0.10% |
| NaOH | 0.83% |
| Methocell E5 (Hydroxypropyl Methyl Cellulose) | 2.36% |

### Example 8

### Comparative Adhesion Tests on Mucosa

The adhesion times on gastric mucosa of 4 samples (1 cm²) of edible films having the following formulation (Reference film):

| | |
|---|---|
| Protanal GP 2650 (sodium alginate) | 30.33% |
| Avicel ph 105 (microcrystalline cellulose) | 6.30% |
| Pisane M9 (Vegetal proteins from pea) | 11.42% |
| Sorbitol 70% | 21.66% |
| Zemea | 5.51% |
| Tween 80 (Polysorbate 80 stabilizer) | 9.45% |
| Renovhyal (ialuronic acid) | 0.06% |
| Taurina | 0.59% |
| PVP K90 | 8.27% (5.51 + 2.76) |
| Xilitol | 0.59% |
| Propoli | 0.59% |
| Symrise | 0.10% |
| NaOH | 0.83% |
| Methocell E5 (Hydroxypropyl Methyl Cellulose) | 2.36% |

and a thickness of 50 urn were compared with 4 samples of a film of Example 7

### Experimental Conditions

### Animals Characterisation

### Species: White Mice

### Strain: CD1

### N.: 5

Weight: 20-25 g at the starting of the test
Age: 8 weeks
Sex: female

### Caging

Each mouse was caged in cages of mm 160×270×370 (L×W×H).

Automatic control assured 12 hours light, 12 hours dark.

Room temperature and humidity were regulated by a conditioning plant.

### Cleaning and Disinfection

The cages and the housing room were cleaned before the animals were accommodated, then cleaning and disinfection were performed daily.

### Feeding

Animals were fed with standard pelletized diet, provided by Harlan Italy.

### Watering

Animals are watered with purified water, available ad libitum and distributed via an automatic watering system.

### Animal Identification

Animals selected for the study were identified via a numbered plastic mark applied to the right ear. Cages were identified with a tag.

### Animal Selection

Animals used in the study were randomly assigned to the different treatment groups at the beginning of the study.

### Materials and Methods

Gastric mucosa has been obtained from 8 week old mice. Immediately after sacrifice the stomach has been removed open longitudinally, the mucosa exposed and fixed on an hard by the use of needles. On the mucosa surface “the film of Example 7” and “Reference film” have been allowed to adhere, after that the samples have been dipped in a saline solution at 37° C. and gently steered for 3 and 10 minutes. After each time the samples were collected and frizzed at −20° C. to be cut at cryostat. Single section, 15 micron thick, have been collected and observed, unstained, under light microscopy. Images have been recorded using a digital camera.

### 2. Results

It was observed an initial ability of both the films to adhere to a wet mucosa. After 3 minutes of washing both films are still present on the mucosa surface, demonstrating their muco-adhesion ability. The “reference film” lose about 30% of thickness if compared to T0. At the contrary the “film of Example 7” shows a thickens increase. After 10 minutes of washing the “film of Example 7” is still entire and visible on the mucosa surface, on the contrary the “Reference film” is limited to a partially thin layer on the mucosa.

### Dissolution Times

The dissolution time has been evaluated for both the films (Film of EXAMPLE 7 and Reference film). Both films, in their free form, have been dip in a saline solution at 37° C. and gently steered.

The “Reference film” (only PVP) dissolved within 20 minutes (the evaluation has been performed in triplicate) versus about 110 minutes of the film of Example 7 including synergic combination of PVP and Carbopol (the evaluation has been performed in triplicate).

### Example 9

### Comparative Adhesion Tests on Mucosa

The adhesion times on gastric mucosa of 4 samples of edible films having the following formulation (Reference film 9):

| | |
|---|---|
| Protanal GP 2650 (sodium alginate) | 30.33% |
| Avicel ph 105 (microcrystalline cellulose) | 6.30% |
| Pisane M9 (Vegetal proteins from pea) | 11.42% |
| Sorbitol 70% | 21.66% |
| Zemea | 5.51% |
| Tween 80 (Polysorbate 80 stabilizer) | 9.45% |
| Renovhyal (ialuronic acid) | 0.06% |
| Taurina | 0.59% |
| Xilitol | 0.59% |
| Carbopol 091 P NF | 8.27% (2.76 + 5.51) |
| Propoli | 0.59% |
| Symrise | 0.10% |
| NaOH | 0.83% |
| Methocell E5 (Hydroxypropyl Methyl Cellulose) | 2.36% |

and a thickness of 50 μm were compared with 4 samples of a film having the formulation of EXAMPLE 10:

### Experimental Conditions

### Animals Characterisation

### Species: White Mice

### Strain: CD1

### N.: 5

Weight: 20-25 g at the starting of the test
Age: 8 weeks
Sex: female

### Caging

Each mouse was caged in cages of mm 160×270×370 (L×W×H).

Automatic control assured 12 hours light, 12 hours dark.

Room temperature and humidity were regulated by a conditioning plant.

### Cleaning and Disinfection

The cages and the housing room were cleaned before the animals were accommodated, then cleaning and disinfection were performed daily.

### Feeding

Animals were fed with standard pelletized diet, provided by Harlan Italy.

### Watering

Animals are watered with purified water, available ad libitum and distributed via an automatic watering system.

### Animal Identification

Animals selected for the study were identified via a numbered plastic mark applied to the right ear. Cages were identified with a tag.

### Animal Selection

Animals used in the study were randomly assigned to the different treatment groups at the beginning of the study.

### Materials and Methods

Gastric mucosa has been obtained from 8 week old mice. Immediately after sacrifice the stomach has been removed open longitudinally, the mucosa exposed and fixed on an hard by the use of needles. On the mucosa surface “the film of Example 10” and “Reference film 9” have been allowed to adhere, after that the samples have been dipped in a saline solution at 37° C. and gently steered for 3 and 10 minutes. After each time the samples were collected and frizzed at −20° C. to be cut at cryostat. Single section, 15 micron thick, have been collected and observed, unstained, under light microscopy. Images have been recorded using a digital camera.

### 2. Results

It was observed an initial ability of both the films of Example 10 and the “reference film” to adhere to a wet mucosa.

After 3 minutes of washing both films are still present on the mucosa surface, demonstrating their muco-adhesion ability. The “reference film 9” lose about 30% of thickness if compared to T0. At the contrary the “film of Example 10” shows a thickens increase. After 10 minutes of washing the “film of EXAMPLE 10” is still entire and visible on the mucosa surface, on the contrary the “reference film 9” is limited to a partially thin layer on the mucosa.

### Dissolution Times

Dissolution time has been evaluated for both the films (Reference film 9 and EXAMPLE 10). Both films, in their free form, have been dip in a saline solution at 37° C. and gently steered.

The “reference film 9” (only CARBOPOL) dissolved within 35 minutes (the evaluation has been performed in triplicate) versus about 110 minutes of the film of Example 10 including synergistic combination of PVP and Carbopol (the evaluation has been performed in triplicate).

The films of Example 7 and Example 10 ie those with synergistic mixture of PVP and Carbopol, proved to be the most effective to prolong the dissolution time and to remain firmly sticked to the mucosal surface.

The formulations with PVP and Carbopol together also showed more elasticity, plasticity and patient compliance.

### Example 10

Mucoadhesive edible polymeric film of the invention having a thickness of 50 μm and the following composition:

| | |
|---|---|
| Protanal GP 2650 (sodium alginate) | 30.33% |
| Avicel ph 105 (microcrystalline cellulose) | 6.30% |
| Pisane M9 (Vegetal proteins from pea ) | 11.42% |
| Sorbitol 70% | 21.66% |
| Zemea | 5.51% |
| Tween 80 (Polysorbate 80) | 9.45% |
| Renovhyal (ialuronic acid) | 0.06% |
| Taurina | 0.59% |
| PVP K90 | 5.51% |
| Xilitol | 0.59% |
| Carbopol 971 P NF | 2.76% |
| Propoli | 0.59% |
| Symrise | 0.10% |
| NaOH | 0.83% |
| Methocell E5 (Hydroxypropyl Methyl Cellulose) | 2.36% |

1. A mucoadhesive edible slow dissolving film for treating or protecting oral ulcerations comprising a salt of alginic acid, an edible cellulose derivative, and an edible polymer delaying the dissolution in a physiological fluid comprising a mixture of polyvinylpyrrolidone and a carboxyvinyl polymer.

2. (canceled)

3. The mucoadhesive edible slow dissolving film of claim 1, wherein the salt of alginic acid is in an amount of 20 to 65% by weight, the edible cellulose derivative is in amount of 0.1 to 25% by weight, and the edible polymer retarding the dissolution in a physiological fluid of the film is in amount of 0.1 to 25% by weight.

4. The mucoadhesive edible slow dissolving film according to claim 1 wherein the salt of alginic acid is sodium alginate.

5. The mucoadhesive edible slow dissolving film according to claim 1 wherein the cellulose derivative is selected from the group comprising hydroxypropylmethyl cellulose, methyl cellulose, hydroxypropyl cellulose, hydroxylethyl cellulose, carboxymethyl cellulose, ethylhydroxyethyl cellulose, cellulose acetate butyrate, cellulose acetate phthalate, hydroxypropyl-methylcellulose, ethyl-methylcellulose, microcrystalline cellulose and mixtures thereof.

6. The mucoadhesive edible slow dissolving film according to claim 1 further comprising an edible vegetal protein.

7. The mucoadhesive edible slow dissolving film according to claim 1 further containing water in an amount such that its activity (Aw), is of 0.05 to 0.6.

8. The mucoadhesive edible slow dissolving film according to claim 1 having a dissolution time of 20 minutes to 2 hours in water at 37° C.

9. The mucoadhesive edible slow dissolving film according to claim 1 wherein the film has a thickness of 50 to 200 μm.

10. The mucoadhesive edible slow dissolving film according to claim 1 further comprising a cicatrizing ingredient and/or a re-epithelialization ingredient.

11. The mucoadhesive edible film of claim 10 wherein the cicatrizing and re-epithelialization ingredient is hyaluronic acid.

12. The mucoadhesive edible film according to claim 1, further comprising an antiseptic agent.

13. The mucoadhesive edible film according to claim 1 further comprising a local immune stimulating agent.

14. A method for the prevention or treatment of an oral ulceration comprising the application of an oral mucosa of an effective amount of a mucoadhesive edible slow dissolving film comprising a salt of alginic acid, an edible cellulose derivative, and an edible polymer delaying the dissolution in a physiological fluid comprising a mixture of polyvinylpyrrolidone and a carboxyvinyl polymer.

15. The method according to claim 14 wherein said oral ulceration is a lesion of the oral mucosa or gingiva, an aphthous ulcer or an herpetic ulcer.

16. The method according to claim 14 wherein the mucoadhesive edible slow dissolving film when applied on an oral mucosa or gingiva makes a barrier protecting an ulceration or a lesion of the oral mucosa or gingiva from mechanical, chemical or infective agents.

17. The mucoadhesive edible film according to claim 1 further comprising a natural gum.
